(19) United States
(12) Patent Application Publication
(10) Pub. No.: US 2009/0198080 A1
(43) Pub. Date: Aug. 6, 2009

(54) METHOD FOR THE PREPARATION OF ARYL ETHERS

(75) Inventors: Mateusz Cebula, Portage, MI (US); Kevin Edward Henegar, East Lyme, CT (US)

Correspondence Address: PFIZER INC.;PATENT DEPARTMENT, Bld 114 M/S 114, EASTERN POINT ROAD, GROTON, CT, 06340, US

(73) Assignee: Pfizer Inc

(21) Appl. No.: 12/422,494

(22) Filed: Apr. 13, 2009

Related U.S. Application Data
(63) Continuation of application No. 11/060,172, filed on Feb. 17, 2005.
(60) Provisional application No. 60/546,495, filed on Feb. 20, 2004.

(51) Int. Cl. C07F 7/08; C07C 41/03; C07C 213/04

(52) U.S. Cl. 556/428; 568/644; 564/349

(57) ABSTRACT

The invention comprises an enantioselective method of preparing a compound of formula (IV):

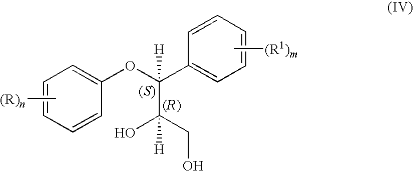

wherein n, m, R and R¹ are as defined herein.

Use of the compounds of formula (IV) in a method of preparing compounds of formula (IX) and novel intermediates useful in the method are also described.

This application is a United States utility application, which claims the benefit of priority to United State Provisional Application No. 60/546,495, filed Feb. 20, 2004.

### FIELD OF THE INVENTION

The present invention relates to an improved method for preparing certain aryl ethers. The invention also relates to intermediates useful in the method, and to methods for preparing such intermediates.

### BACKGROUND TO THE INVENTION

U.S. Pat. No. 4,229,449 discloses compounds of formula (A)

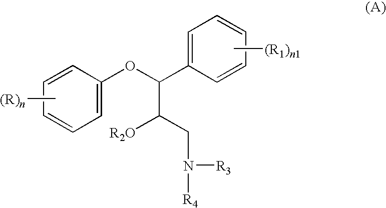

wherein
n and n1 are, independently, 1, 2 or 3;
each or the groups R and R₁, which may be the same or different, is hydrogen; halogen; halo-C₁-C₆alkyl; hydroxy; C₁-C₆alkoxy; C₁-C₆alkyl optionally substituted; aryl-C₁-C₆alkyl optionally substituted; aryl-C₁-C₆alkoxy optionally substituted; —NO₂; NR₅R₆ wherein R₅ and R₆ are, independently, hydrogen or C₁-C₆ alkyl, or two adjacent R groups or two adjacent R₁ groups, taken together, form a —O—CH₂—O— radical;
R₂ is hydrogen; C₁-C₁₂alkyl optionally substituted, or aryl-C₁-C₆alkyl;
each of the groups R₃ and R₄, which may be identical or different, is hydrogen, C₁-C₆ alkyl optionally substituted, C₂-C₄ alkenyl, C₂-C₄ alkynyl, aryl-C₁-C₄ alkyl optionally substituted, C₃-C₇ cycloalkyl optionally substituted, or R₃ and R₄ with the nitrogen atom to which they are bounded form a pentatomic or hexatomic saturated or unsaturated, optionally substituted, heteromonocyclic radical optionally containing other heteroatoms belonging to the class of O, S and N;
or R₂ and R₄, taken together, form a —CH₂—CH₂— radical;
or a pharmaceutically acceptable salt thereof.

The compounds are disclosed to possess antidepressant activity.

In particular, U.S. Pat. No. 4,229,449 discloses the compound: 2-[α-(2-ethoxyphenoxy)benzyl]morpholine:

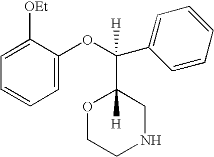

and pharmaceutically acceptable salts thereof, which possess useful antidepressant properties. This compound is also known as reboxetine.

Reboxetine does not act like most antidepressants. Unlike tricyclic antidepressants, and even selective serotonin reuptake inhibitors (SSRIs), reboxetine is ineffective in the 8-OH-DPAT hypothermia test, indicating that reboxetine is not a SSRI. See Brian E. Leonard, “Noradrenaline in basic models of depression.” European-Neuropsychopharmacol, 7 Suppl. 1 pp. S11-6 and S71-3 (April 1997), incorporated herein in its entirety by reference thereto. Reboxetine is a selective norepinephrine reuptake inhibitor, with only marginal serotonin and no dopamine reuptake inhibitory activity. Reboxetine displays no anticholinergic binding activity in different animal models, and is substantially devoid of monoamine oxidase (MAO) inhibitory activity.

Many organic compounds exist in optically active forms, i.e., they have the ability to rotate the plane of plane-polarized light. In describing an optically active compound the prefixes R and S are used to denote the absolute configuration of the molecule about its chiral center(s). The prefixes D and L, or (+) or (−), designate the sign of rotation of plane-polarized light by the compound, with L or (−) meaning that the compound is levorotatory. In contrast, a compound prefixed with D or (+) is dextrorotatory. There is no correlation between nomenclature for the absolute stereochemistry and for the rotation of an enantiomer. Thus, D-lactic acid is the same as (−)-lactic acid, and L-lactic acid is the same as (+)-lactic acid. For a given chemical structure, each of a pair of enantiomers are identical except that they are non-superimposable mirror images of one another. A specific stereoisomer may also be referred to as an enantiomer, and a mixture of such isomers is often called an enantiomeric, or racemic, mixture.

When two chiral centers exist in one molecule, there are four possible stereoisomers: (R,R), (S,S), (R,S), and (S,R). Of these, (R,R) and (S,S) are an example of a pair of enantiomers (mirror images of each other), which typically share chemical properties and melting points just like any other enantiomeric pair. The mirror images of (R,R) and (S,S) are not, however, superimposable on (R,S) and (S,R). This relationship is called diastereoisomeric, and the (S,S) molecule is a diastereoisomer of the (R,S) molecule, whereas the (R,R) molecule is a diastereoisomer of the (S,R) molecule.

Chemically, reboxetine has two chiral centres and, therefore, exists as two enantiomeric pairs of diastereomers, the (R,R) and (S,S) enantiomeric pair and the (R,S) and (S,R) enantiomeric pair. Currently, reboxetine is commercially available only as a racemic mixture of enantiomers, (R,R) and (S,S) in a 1:1 ratio, and reference herein to the generic name “reboxetine” refers to this enantiomeric, or racemic, mixture. Reboxetine is commercially sold under the trade names of EDRONAX™, PROLIFT™, VESTRA™, and NOREBOX™.

It is now known (see WO 01/01973, incorporated in its entirety by reference) that the (S,S)-enantiomer of reboxetine possesses greatly improved selectivity for norepinephrine reuptake over serotonin reuptake. Accordingly, WO 01/01973 discloses a method of selectively inhibiting reuptake of norepinephrine, the method comprising the step of administering a therapeutically effective amount of a composition to an individual, the composition comprising a compound having a pharmacological selectivity of serotonin (Kᵢ)/norepinephrine (Kᵢ) of at least about 5000. The document further discloses a number of novel uses of (S,S)-reboxetine, including use of (S,S)-reboxetine in the treatment of chronic pain, peripheral neuropathy, incontinence (including stress incontinence, genuine stress incontinence, and mixed incontinence), fibromyalgia and other somatoform disorders, and migraine headaches.

U.S. Pat. Nos. 5,068,433 and 5,391,735, as well as GB-A-2162517, disclose processes and intermediates useful for preparing single diastereomers of compounds of formula VIb:

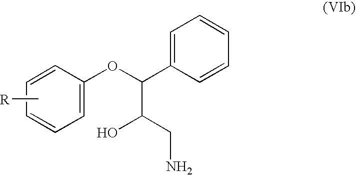

wherein R is C₁-C₆ alkoxy or tri-halomethyl. These diastereomers are disclosed to be useful intermediates for preparing compounds of formula A, including reboxetine. The processes disclosed in these patents and in U.S. Pat. No. 4,229,449, however, are inefficient and provide a low overall yield of compounds of formula A when carried out on a commercial scale. Additionally the processes require the use of expensive reagents and require significant production times. Thus, it is not economical to prepare compounds of formula A on a commercial scale using the processes disclosed in these patents.

WO 00/39072 provides a method for preparing an amine of formula VIIa:

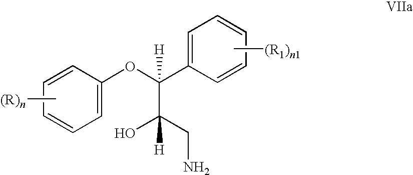

comprising:
a) oxidizing an optionally substituted trans-cinnamyl alcohol to give an intermediate epoxide of formula Ia:

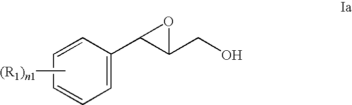

b) reacting the epoxide with an optionally substituted phenol to give a diol of formula IIa:

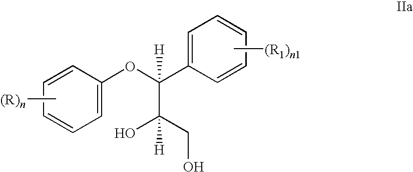

c) reacting the diol with a silylating reagent to give an alcohol of formula IIIa:

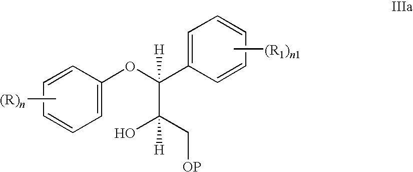

wherein P is a silyl-linked radical;
d) reacting the alcohol of formula IIIa with reactive derivative of a sulfonic acid to give a compound of formula IVa:

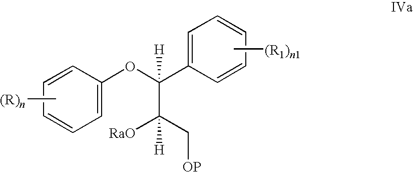

wherein Ra is a residue of a sulfonic acid;
e) removing P from the compound of formula IVa to give an alcohol of formula Va:

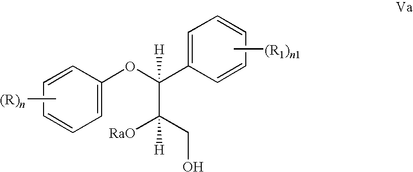

f) displacing the sulfonyloxy group to give an epoxide of formula VIa:

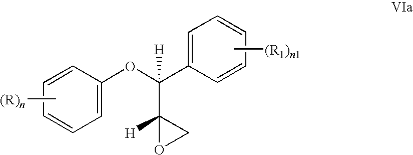

and
g) reacting the epoxide with ammonia to give the compound of formula VIIa.

WO 00/39072 also provides a method for the preparation of racemic reboxetine from the above amine, comprising:

h) reacting a compound of formula VIIa:

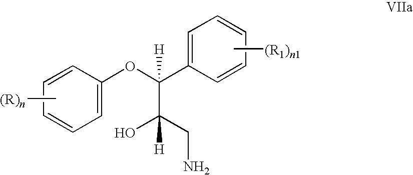

with a carboxylic acid of formula HOOCCH₂L or a reactive derivative thereof, wherein L is a leaving group, to give an amide of formula VIIIa:

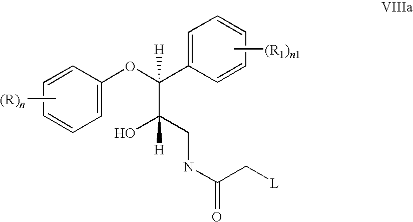

i) reacting the compound of formula VIIIa to give a compound of formula IXa:

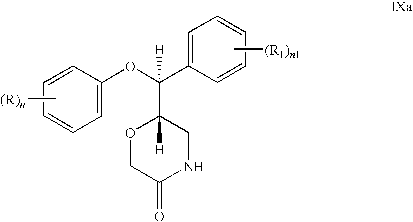

and
j) reducing the compound of formula IXa to give a corresponding compound of the following formula:

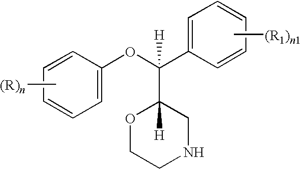

In the above formulae, R, R₁, n and n1 are as defined in U.S. Pat. No. 4,229,449 referred to above.

The above publications all disclose methods for producing reboxetine and related compounds in the form of a racemic mixture of the (R,R)- and (S,S)-enantiomers. A further resolution step is required in order to isolate the more potent (S,S)-enantiomer.

An alternative synthesis of (S,S)-reboxetine is described in GB-A-2167407. This document discloses a chiral synthesis of (S,S)-reboxetine starting from chiral phenylglycidic acid. However, no adequate chiral syntheses of the phenylglycidic acid exist so the chiral acid must be prepared by resolution, which is inefficient. The subsequent reduction to phenylglycidol is low yielding. Following this step, the synthesis described in GB-A-2167407 parallels the racemic synthesis: as remarked above, this synthesis exhibits poor selectivity and is low yielding and inefficient.

It would be desirable to provide an enantioselective synthesis of (S,S)-aryl ethers useful in the production of (S,S)-reboxetine which avoids the production of the unwanted (R,R)-enantiomer and allows (S,S)-reboxetine to be produced more efficiently and in a greater yield and purity than is allowed by the syntheses of the prior art.

We have surprisingly found that a novel synthesis according to the present invention allows intermediates useful in the synthesis of (S,S)-reboxetine to be produced enantioselectively, efficiently and in high yield and purity.

### SUMMARY OF THE INVENTION

In one aspect, the invention provides a method of preparing a compound of formula (IV):

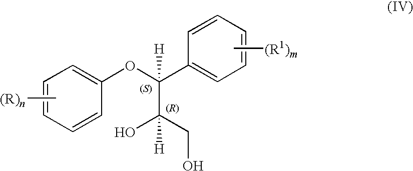

wherein
n and m are, independently, 0 or an integer from 1 to 5;
each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆alkyl wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆alkoxy wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical; comprising:
   (a) asymmetric epoxidation of a compound of formula (I):

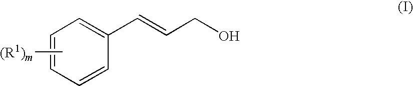

wherein R¹ and m are as defined above, with an oxidising agent in the presence of an optically active compound, to give a compound of formula (II):

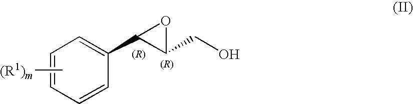

wherein R¹ and m are as defined above, followed by
(b) reaction of the compound of formula (II) with a compound of formula (III):

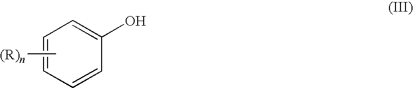

wherein R and n are as defined above, in the presence of a base;
wherein the method is carried out without isolation of the compound of formula (II).

Surprisingly, we have found that carrying out steps (a) and (b) sequentially without isolating the compound of formula (II) allows the compound of formula (IV) to be produced enantioselectively and in good yield and purity. This is contrary to what would have been expected as it would have been thought necessary to isolate the compound of formula (II) from the agents used in its synthesis and work-up: it would have been expected that the presence of these agents would interfere with the synthesis and purification of the compound of formula (IV).

The invention further provides a method of preparing a compound of formula (IX):

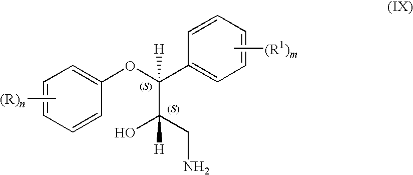

wherein R, R¹, n and m are as defined above, comprising:
(a) preparing a compound of formula (IV) according to the above method;
(b) reaction of the compound of formula (IV) with a silylating agent to produce a compound of formula (V):

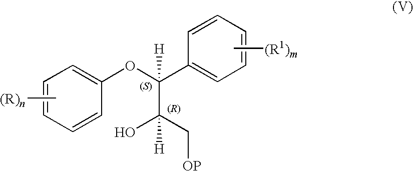

wherein R, R¹, n and m are as defined above, and P is a silyl protecting group;
(c) reaction of the compound of formula (V) with a sulfonylating agent of formula R′SO₂X, wherein R′ is a sulfonic acid residue and X is a leaving group, to produce a compound of formula (VI):

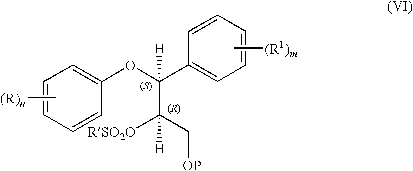

wherein R, R¹, n and m are as defined above, P is as defined above, and R′ is a sulfonic acid residue;
(d) removal of the silyl protecting group to produce a compound of formula (VII):

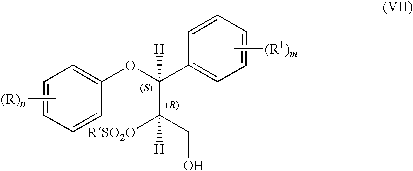

wherein R, R¹, n and m are as defined above, and R′ is as defined above;
(e) displacement of the sulfonyloxy group to give a compound of formula (VIII):

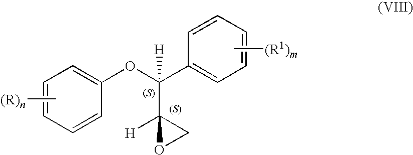

wherein R, R¹, n and m are as defined above; and
(f) reaction of the compound of formula (VIII) with ammonia or an ammonium compound to give the compound of formula (IX).

Preferably, the method is carried out without isolating the compounds of formulae (V), (VI), (VII) and (VIII).

In another aspect, the invention provides a compound of formula (V):

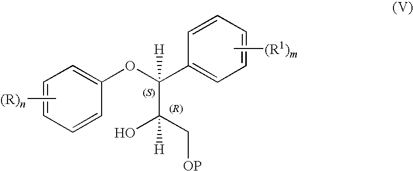

wherein R, R¹, n and m are as defined above, and P is a silyl protecting group.

In a further aspect, the invention provides a compound of formula (VI):

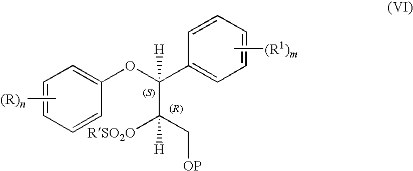

wherein R, R¹, n, m, P and R′ are as defined above.

### DETAILED DESCRIPTION OF PREFERRED EMBODIMENTS

In the compounds of formulae (I) through (IX), each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆alkyl wherein the aryl ring is optionally substituted by one or more substituents selected from C₁-C₆alkyl, halogen, halo-C₁-C₆alkyl, hydroxy, C₁-C₆ alkoxy, and NR⁵R⁶ wherein R⁵ and R⁶ are, independently, hydrogen or C₁-C₆ alkyl; aryl-C₁-C₆ alkoxy wherein the aryl ring is optionally substituted by one or more substituents selected from C₁-C₆alkyl, halogen, halo-C₁-C₆alkyl, hydroxy, C₁-C₆ alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O-radical; and n and m are, independently, 0 or an integer from 1 to 5.

The term “alkyl” means a straight or branched chain saturated hydrocarbon group containing 1 to 6 carbon atoms.

The term “haloalkyl” means an alkyl group, as defined above, which is substituted by one or more halogen atoms.

The term “alkoxy” means “alkyl-O—”, wherein the alkyl group is as defined above.

The term “aryl” means a phenyl or naphthyl group.

Suitably, the groups R and R¹, which may be the same or different, are selected from hydroxy and C₁-C₆alkoxy. Preferably, R is methoxy or ethoxy, more preferably ethoxy.

Preferably, n is 1 or 2, more preferably 1.

Preferably, m is 0 or 1, more preferably 0.

In especially preferred embodiments, n is l, m is 0 and R is ethoxy at the 2-position of the phenyl ring.

### Step 1

The process of the present invention begins with asymmetric epoxidation of a compound of formula (I) with an oxidising agent in the presence of an optically active compound, to give the (R,R) enantiomer of a compound of formula (II).

The asymmetric epoxidation of the compound of formula (I) is achieved using an oxidising agent in the presence of an optically active compound. The precise nature of the oxidising agent and the optically active compound are not critical provided they are capable of achieving epoxidation of the olefin in an asymmetric manner to give the (R,R) enantiomer of a compound of formula (II). The oxidising agent may itself be optically active, in which case there is no need to supply a separate optically active compound.

Examples of suitable oxidising agents include hydroperoxides such as t-butyl hydroperoxide, cumene hydroperoxide, α,α-dimethylheptyl hydroperoxide, bis diisobutyl-2,5-dihydroperoxide, 1-methylcyclohexyl hydroperoxide, or cyclohexyl hydroperoxide, or dioxiranes, particularly chiral dioxiranes of the type described by Yian Shi et al, J. Am. Chem. Soc. 1997, 119, 11224-11235. In the case of chiral dioxiranes, as the dioxirane is itself chiral there is no need to supply a separate optically active compound.

Examples of suitable optically active compounds include salts and esters of optically active organic acids, particularly tartaric acid esters. A particularly preferred example of an optically active compound is (−)-diisopropyl tartrate.

The reaction may optionally be carried out in the presence of further reagents, examples of which include titanium alkoxides such as titanium methoxide, ethoxide, n-propoxide, isopropoxide, n-, s-, l-, or t-butoxide. Titanium isopropoxide is preferred.

It is particularly preferred that the asymmetric epoxidation is carried out under the conditions described in J. Am. Chem. Soc., 1980, 102, 5974-5976 (“Sharpless asymmetric epoxidation”), wherein the oxidising agent is t-butyl hydroperoxide, the optically active compound is (−)-diisopropyl tartrate and the reaction is carried out in the presence of titanium isopropoxide. Further information on the procedure can be found in U.S. Pat. No. 4,471,130 and in K. B. Sharpless et al, J. Am. Chem. Soc. 1987, 110, 5765-5780.

The reaction is normally and preferably carried out in the presence of a solvent, the nature of which is not especially critical provided it is inert to the reaction and is capable of dissolving the reactants at least to some extent. Examples of suitable solvents include aliphatic hydrocarbons such as pentanes, hexanes, heptanes and octanes, nonanes, or decanes, aromatic hydrocarbons such as benzene, toluene and xylenes, halogenated hydrocarbons such as methylene chloride, chloroform and 1,2-dichloroethane, ethers such as methyl tert-butyl ether, and mixtures thereof. It is preferred that the solvent is a mixture of methylene chloride and aliphatic hydrocarbons.

The reaction temperature depends on various factors such as the nature of the reagents and the solvent. However, it is typically from −50° C. to room temperature, and preferably −30° C. to 0° C.

The reaction time depends on various factors such as the nature of the reagents, the solvent and the temperature. However, it is typically from 10 minutes to 24 hours, preferably from 30 minutes to 12 hours, and more preferably 1 to 6 hours.

The reaction is preferably carried out under anhydrous conditions. Molecular sieves are preferably present in the reaction mixture to absorb any water present.

After completion of the reaction, a quenching agent is typically added in order to quench any excess oxidising agent present. The nature of the quenching agent is important, since most conventional quenching agents are either ineffective or the product is unstable in their presence. Preferred quenching agents are (C₁-C₆) alkyl phosphites such as trimethyl phosphite and triethyl phosphite. Triethyl phosphite is especially preferred.

The compound of formula (II) is not isolated from the reaction mixture. Rather, the reaction mixture containing the compound of formula (II) and the quenching agent is reacted directly with the compound of formula (III) in the next step.

### Step 2

In the next step the compound of formula (II) is reacted with a compound of formula (III) in the presence of a base.

The nature of the base is not especially critical provided that it is capable of acting as a base. Examples of suitable bases include alkali metal hydroxides such as lithium hydroxide, sodium hydroxide, potassium hydroxide, rubidium hydroxide and caesium hydroxide, tetra(C₁-C₆)alkylammonium hydroxides such as tetramethylammonium hydroxide and tetraethylammonium hydroxide, alkali metal carbonates such as lithium carbonate, sodium carbonate, potassium carbonate, rubidium carbonate and caesium carbonate, and organic amines such as trimethylamine and triethylamine. Alkali metal hydroxides are preferred and sodium hydroxide is especially preferred.

The reaction is normally and preferably carried out in the presence of a solvent, the nature of which is not especially critical provided it is inert to the reaction and is capable of dissolving the reactants at least to some extent. Examples of suitable solvents include water, amides such as dimethylformamide, DMA, and NMP, aliphatic hydrocarbons such as pentanes, hexanes, heptanes and octanes, aromatic hydrocarbons such as benzene, toluene and xylenes, halogenated hydrocarbons such as methylene chloride, chloroform and 1,2-dichloroethane, ethers such as tetrahydrofuran, methyl tert-butyl ether, dioxane, diethyl ether, diisopropyl ether, and dimethoxyethane, and mixtures thereof. It is preferred that the solvent is a biphasic mixture of methylene chloride and water.

The reaction is preferably carried out in the presence of a phase transfer catalyst, the function of which is to transfer a basic anion such as hydroxide ion into the organic layer. Examples of suitable phase transfer catalysts include tetra(C₁-C₆)alkylammonium and benzyltri(C₁-C₆)alkylammonium salts such as tetra-n-butylammonium chloride and benzyltriethylammonium chloride. Tetra-n-butylammonium chloride is preferred.

The reaction temperature depends on various factors such as the nature of the reagent, the base and the solvent. However, it is typically from room temperature to 80° C., and more preferably from 35° C. to 70° C.

The reaction time depends on various factors such as the nature of the reagents, the solvent and the temperature. However, it is typically from 10 minutes to 24 hours, preferably from 30 minutes to 12 hours, and more preferably 1 to 6 hours.

After completion of the reaction, the compound of formula (IV) is isolated from the reaction mixture by a conventional method. For example, the compound may be extracted using an organic solvent, the organic layer may be washed with an aqueous solution such as water, sodium hydroxide or sodium chloride in order to remove any ionic species present, filtered to remove any solid matter, and concentrated to remove the solvent. The product may further be purified by conventional methods such as crystallisation or column chromatography.

### Step 3

In this step the aryl ether of formula (IV) is silylated to produce a compound of formula (V). The conversion is achieved by reaction with a silylating agent in the presence of a base.

The precise nature of the silyl protecting group P attached in this step is not critical to the present invention provided that it is usually capable of protecting a hydroxyl group. Preferably, the silyl protecting group is capable of protecting a primary hydroxyl group in the presence of a secondary hydroxyl group. Examples of suitable silyl groups include trimethylsilyl, tert-butyldimethylsilyl, triethylsilyl, triisopropylsilyl and tert-butyldiphenylsilyl groups, of which the trimethylsilyl group is preferred.

The silylating agent is typically a silyl halide, preferably a chloride.

The nature of the base is not especially critical provided that it is capable of acting as a base. Examples of suitable bases include alkali metal hydroxides such as lithium hydroxide, sodium hydroxide, potassium hydroxide, rubidium hydroxide and caesium hydroxide, tetra(C₁-C₆)alkylammonium hydroxides such as tetramethylammonium hydroxide and tetraethylammonium hydroxide, alkali metal carbonates such as lithium carbonate, sodium carbonate, potassium carbonate, rubidium carbonate and caesium carbonate, and organic amines such as trimethylamine, triethylamine, pyridine, methyldiethylamine, dimethylethylamine, tri n-propylamine, triisopropylamine, diisopropylethylamine, tributylamines, and higher trialkylamines, picolines, lutidines, and collidines, 2-methyl-5-ethylpyridine (lonza pyridine), 2,6-di-t-butylpyridine, 2,6-di-t-butyl-4-methylpyridine and alkyl quinolines and isoquinolines, N-methylpiperidine, N-methylpyrrolidine, N-methylmorpholine, and higher alkyl analogues of these compounds. Organic amines are preferred and triethylamine is especially preferred.

The reaction is normally and preferably carried out in the presence of a solvent, the nature of which is not especially critical provided it is inert to the reaction and is capable of dissolving the reactants at least to some extent. Examples of suitable solvents include amides such as dimethylformamide, aliphatic hydrocarbons such as pentanes, hexanes, heptanes and octanes, aromatic hydrocarbons such as benzene, toluene and xylenes, halogenated hydrocarbons such as methylene chloride, chloroform and 1,2-dichloroethane, ethers such as diethyl ether, methyl tert-butyl ether, tetrahydrofuran, diisopropyl ether, dimethoxyethane and dioxane, esters such as ethyl acetate, and mixtures thereof. It is preferred that the solvent is a halogenated hydrocarbon, especially methylene chloride.

The reaction temperature depends on various factors such as the nature of the silylating agent, the base and the solvent. However, it is typically from −78° C. to room temperature, and is preferably −50° C. to −10° C.

The reaction time depends on various factors such as the nature of the reagents, the solvent and the temperature. However, it is typically from 5 minutes to 2 hours, and preferably from 10 minutes to 1 hour.

The compounds of formula (V) are new and therefore form part of the present invention.

The compound of formula (V) is not normally isolated from the reaction mixture. Rather, a solution containing the compound of formula (V) is reacted directly with the sulfonylating agent in the next step.

### Step 4

In this step the silyl-protected compound of formula (V) is sulfonylated to produce a compound of formula (VI). This conversion is achieved by reaction of the compound of formula (V) with a sulfonylating agent of formula R′SO₂X in the presence of a base.

The nature of the sulfonylating agent is not especially critical to the present invention, provided it can usually be used to sulfonylate a hydroxy group. Examples of the sulfonic acid residue R′ include (C₁-C₆) alkyl groups, halo-(C₁-C₆)alkyl groups and phenyl groups optionally substituted with 1 to 3 (C₁-C₆) alkyl groups or halogen atoms, and preferred examples include methyl, ethyl, trifluoromethyl, phenyl and p-tolyl. Examples of the leaving group X include halogen atoms and sulfonyloxy groups of formula R′O wherein R′ is one of the sulfonic acid residues mentioned above, and preferred are halogen atoms. Preferred examples of sulfonylating agents include methanesulfonyl chloride, benzenesulfonyl chloride, p-toluenesulfonyl chloride and p-toluenesulfonic anhydride, and methanesulfonyl chloride is especially preferred.

The nature of the base is not especially critical provided that it is capable of acting as a base. Examples of suitable bases include organic amines such as trimethylamine, triethylamine, pyridine, methyldiethylamine, dimethylethylamine, tri-n-propylamine, triisopropylamine, diisopropylethylamine, tributylamines, and higher trialkylamines, picolines, lutidines, and collidines, 2-methyl-5-ethylpyridine (lonza pyridine), 2,6-di-t-butylpyridine, 2,6-di-t-butyl-4-methylpyridine and alkyl quinolines and isoquinolines, N-methylpiperidine, N-methylpyrrolidine, N-methylmorpholine, and higher alkyl analogues of these compounds, and triethylamine is especially preferred.

The reaction is normally and preferably carried out in the presence of a solvent, the nature of which is not especially critical provided it is inert to the reaction and is capable of dissolving the reactants at least to some extent. Examples of suitable solvents include amides such as dimethylformamide, DMA, and NMP, aliphatic hydrocarbons such as pentanes, hexanes, heptanes and octanes, aromatic hydrocarbons such as benzene, toluene and xylenes, halogenated hydrocarbons such as methylene chloride, chloroform and 1,2-dichloroethane, ethers such as diethyl ether, methyl tert-butyl ether, tetrahydrofuran, diisopropyl ether, dimethoxyethane and dioxane, esters such as ethyl acetate, and mixtures thereof. It is preferred that the solvent is a halogenated hydrocarbon, especially methylene chloride.

The reaction temperature depends on various factors such as the nature of the sulfonylating agent, the base and the solvent. However, it is typically from −78° C. to room temperature, and is preferably from −50° C. to −10° C.

The reaction time depends on various factors such as the nature of the reagents, the solvent and the temperature. However, it is typically from 5 minutes to 2 hours, and preferably from 10 minutes to 1 hour.

The compounds of formula (VI) are new and therefore form part of the present invention.

The compound of formula (VI) is not isolated from the reaction mixture. Rather, a solution containing the compound of formula (VI) is reacted directly with the deprotecting agent in the next step.

### Step 5

In this step the silyl protecting group is removed to produce a compound of formula (VII). This step is achieved by reaction with a deprotecting agent.

In this step, the precise nature of the deprotecting agent is not critical provided that it can usually be used to remove a silyl protecting group from a hydroxyl group. Examples of suitable deprotecting agents include acids such as hydrochloric acid, hydrobromic acid, sulphuric acid and acetic acid, and fluoride ion sources such as potassium fluoride and tetra-n-butylammonium fluoride. Acids are preferred and hydrochloric acid is especially preferred.

The reaction is normally and preferably carried out in the presence of a solvent, the nature of which is not especially critical provided it is inert to the reaction and is capable of dissolving the reactants at least to some extent. Examples of suitable solvents include water, alcohols such as methanol and ethanol, amides such as dimethylformamide, aliphatic hydrocarbons such as pentanes, hexanes, heptanes and octanes, aromatic hydrocarbons such as benzene, toluene and xylenes, halogenated hydrocarbons such as methylene chloride, chloroform and 1,2-dichloroethane, ethers such as diethyl ether, methyl tert-butyl ether, tetrahydrofuran, diisopropyl ether, dimethoxyethane and dioxane, and mixtures thereof. It is preferred that the solvent is a biphasic mixture of a halogenated hydrocarbon, especially methylene chloride, and water.

The reaction temperature depends on various factors such as the nature of the deprotecting agent, the base and the solvent. However, it is typically from 0° C. to 50° C., and preferably room temperature.

The reaction time depends on various factors such as the nature of the reagents, the solvent and the temperature. However, it is typically from 2 to 48 hours, preferably 6 to 24 hours, and more preferably 8 to 16 hours.

After completion of the reaction, a solution containing the compound of formula (VII) may be worked up by a conventional method. For example, the solution may be washed with an aqueous solution such as water, sodium bicarbonate or sodium chloride in order to remove any ionic species present, or filtered to remove any solid matter. However, the compound of formula (VII) is not isolated from the solution. Rather, the solution containing the compound of formula (VII) undergoes immediate reaction to displace the sulfonyloxy group in the next step.

### Step 6

In this step, the compound of formula (VII) undergoes intramolecular displacement of the sulfonyloxy group to form a compound of formula (VIII). This is preferably achieved by reaction of the compound of formula (VII) with a base.

The nature of the base is not especially critical provided that it is capable of acting as a base. Examples of suitable bases include alkali metal hydroxides such as lithium hydroxide, sodium hydroxide, potassium hydroxide, rubidium hydroxide and caesium hydroxide, tetra(C₁-C₆)alkylammonium hydroxides such as tetramethylammonium hydroxide and tetraethylammonium hydroxide, and alkali metal carbonates such as lithium carbonate, sodium carbonate, potassium carbonate, rubidium carbonate and caesium carbonate. Alkali metal hydroxides are preferred and sodium hydroxide is especially preferred.

The reaction is normally and preferably carried out in the presence of a solvent, the nature of which is not especially critical provided it is inert to the reaction and is capable of dissolving the reactants at least to some extent. Examples of suitable solvents include water, alcohols such as methanol and ethanol, amides such as dimethylformamide, sulfoxides such as dimethyl sulfoxide, aliphatic hydrocarbons such as pentanes, hexanes, heptanes and octanes, aromatic hydrocarbons such as benzene, toluene and xylenes, halogenated hydrocarbons such as methylene chloride, chloroform and 1,2-dichloroethane, ethers such as dimethoxyethane, diethyl ether, diisopropyl ether, tetrahydrofuran and dioxane, and mixtures thereof. It is preferred that the solvent is a biphasic mixture of methylene chloride and water.

The reaction is preferably carried out in the presence of a phase transfer catalyst, the function of which is to transfer a basic anion such as hydroxide ion into the organic layer. Examples of suitable phase transfer catalysts include tetra(C₁-C₆)alkylammonium and benzyltri(C₁-C₆)alkylammonium salts such as tetra-n-butylammonium chloride and benzyltriethylammonium chloride. Tetra-n-butylammonium chloride is preferred.

The reaction temperature depends on various factors such as the nature of the reagent, the base and the solvent. However, it is typically from 0° C. to the boiling point of the solvent, preferably 10° C. to 40° C., and more preferably room temperature.

The reaction time depends on various factors such as the nature of the reagents, the solvent and the temperature. However, it is typically from 5 minutes to 12 hours, preferably from 10 minutes to 6 hours, and more preferably 30 minutes to 3 hours.

After completion of the reaction, a solution containing the compound of formula (VIII) may be worked up by a conventional method. For example, the solution may be washed with an aqueous solution such as water or sodium chloride in order to remove any ionic species present, filtered to remove any solid matter, or redissolved in another suitable solvent, examples of which are given above. However, the compound of formula (VIII) is not isolated from the solution. Rather, the solution containing the compound of formula (VIII) undergoes immediate reaction with ammonia or an ammonium compound in the next step.

### Step 7

In this step, the epoxide of formula (VIII) is reacted with ammonia or an ammonium compound to produce a compound of formula (IX).

The conversion is achieved by reaction with ammonia, the precise source of which is not especially critical. The ammonia may be gaseous ammonia or ammonia dissolved in a solvent (such as water, methanol or ethanol). Alternatively, the ammonia may be generated in situ by reaction of an ammonium salt (such as ammonium chloride or ammonium acetate) with a base (examples of which are given in Steps 2 and 6).

The reaction is normally and preferably carried out in the presence of a solvent, the nature of which is not especially critical provided it is inert to the reaction and is capable of dissolving the reactants at least to some extent. Examples of suitable solvents include alcohols such as methanol, ethanol and isopropanol, amides such as dimethylformamide, DMA, and NMP, aliphatic hydrocarbons such as pentanes, hexanes, heptanes and octanes, aromatic hydrocarbons such as benzene, toluene and xylenes, ethers such as dimethoxyethane, diethyl ether, diisopropyl ether, tetrahydrofuran and dioxane, and mixtures thereof. It is preferred that the solvent is an alcohol, especially methanol.

The reaction temperature depends on various factors such as the nature of the reagent and the solvent. However, it is typically from room temperature to the boiling point of the solvent, and is preferably from 30° C. to 50° C.

The reaction time depends on various factors such as the nature of the reagents, the solvent and the temperature. However, it is typically from 10 minutes to 12 hours, preferably from 30 minutes to 6 hours, and more preferably 2 to 4 hours.

After completion of the reaction, the compound of formula (IX) is isolated from the reaction mixture by a conventional method. For example, the compound may be extracted using an organic solvent and concentrated to remove the solvent. The compound may be extracted into water as an acid addition salt by the addition of an acid such as hydrochloric acid, neutralised by the addition of a base such as sodium hydroxide, and then extracted using an organic solvent and concentrated to remove the solvent. The product may further be purified by conventional methods such as crystallisation or column chromatography.

### EXAMPLES

The process of the present invention will now be further described with reference to the following Examples. These Examples are intended to illustrate and not limit the scope of the present invention.

### Example 1

### (2R,3R)-2,3-Epoxy-3-phenylpropanol

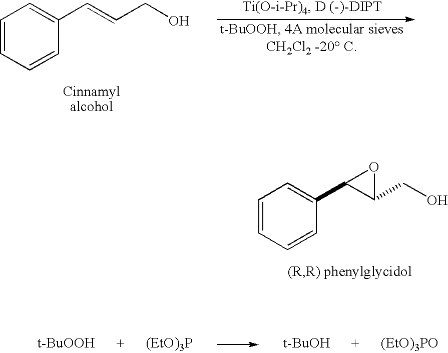

Cinnamyl alcohol (150 g), powdered 4A molecular sieves (60 g), and D-diisopropyl tartrate (39.3 g) were stirred with methylene chloride (2.25 L) and the mixture cooled to −15 to −20° C. Ti(OiPr)₄ (33.2 mL) was added and the reaction mixture was stirred at −20 to −25° C. for 30 minutes. A dry solution of t-butylhydroperoxide in isooctane (500 mL, 4.47 M, KF less than 0.1%) was added over a period of greater than 1 hour maintaining the temperature less than −20° C. After the addition was complete, the mixture was stirred for 3 hours at about −20° C. When the reaction was complete, triethylphosphite (210 mL) was added slowly with cooling maintaining the temperature less than +20° C. The mixture was then filtered over Celite™ to give a solution of the title compound.

### Example 2

### (2R,3S)-3-(2-Ethoxyphenoxy)-2-hydroxy-3-phenylpropanol

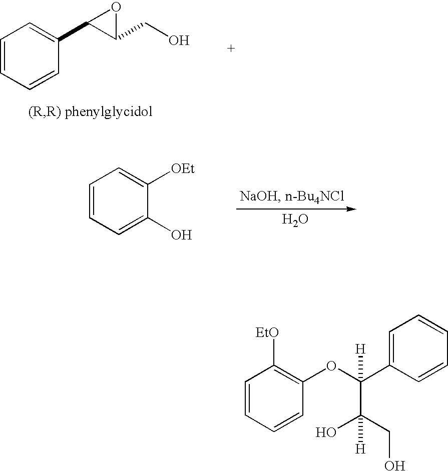

Sodium hydroxide (49.2 g), tetra n-butylammonium chloride (15.5 g), and 2-ethoxyphenol (169.9 g) were dissolved in water (1080 mL). The solution of (2R,3R)-2,3-epoxy-3-phenylpropanol from Example 1 was added and the mixture was heated to about 40° C. internal temperature and the methylene chloride distilled. The internal temperature was gradually increased to 65° C. as the methylene chloride was distilled and heating continued for three hours after completion of the methylene chloride removal. The reaction mixture was cooled to 30° C. Methyl tert-butyl ether (1.5 L) was added and the mixture was stirred for 30 minutes. The phases were allowed to separate and the aqueous phase was removed. The organic layer was washed with 1 M NaOH (2×1 L), water (1 L) and saturated aqueous NaCl solution (1 L). The organic solution was dried by refluxing with a Dean-Stark trap until the KF was about 0.9%. The hot solution was filtered through a 0.2 μm filter. Methyl tert-butyl ether (950 mL) was distilled from the filtrate and isooctane (200 mL) was added. An additional 200 mL of solvent was distilled. Isooctane (200 mL) was added and the solution was cooled until crystals started to form. Once crystallization had started, isooctane (700 mL) was added in three portions over about 1 hour. The slurry was stirred for 15 minutes, then cooled to −20° C., and held at −20° C. for 1 hour. The mother liquor was removed with a stick filter and the solids were washed with isooctane (500 mL). The wash was removed with a stick filter. Methyl tert-butyl ether (300 mL) was added to the solids and the mixture was heated to dissolve the solids. The solution was cooled to about 20-25° C. and isooctane (400 mL) was added in a 50 mL portions over about 30 minutes. The slurry was then cooled slowly to −15° C. The slurry was stirred at −15° C. for one hour and filtered. The crystals were washed with isooctane (300 mL) and then dried on a nitrogen press to give 206 g of the title compound. This material assayed at 97% enantiomeric excess.

### Chiral HPLC Assay:

| | |
|---|---|
| Column: | Chiracel OD-H |
| Mobile phase | 90:10 hexanes:isopropanol |
| Flow | 1 mL/minute |
| Detector | 215 nm |
| Run time | 20 minutes |

### Retention Times

| | |
|---|---|
| (2S,3R)-3-(2-Ethoxyphenoxy)-2-hydroxy-3-phenylpropanol | 8.7 minutes |
| (2R,3S)-3-(2-Ethoxyphenoxy)-2-hydroxy-3-phenylpropanol | 10.8 minutes |

Melting point 91.9-93.4° C.

[α]²⁰_{D} (c=10)+8.33°

¹H NMR (400.13 MHz, CDCl₃) δ 1.51 (t, J=6.6 Hz, 3H), 3.24 (dd, J=3.5 Hz, 9.5 Hz, 1H), 3.33 (d, J=8 Hz, 1H), 3.67 (m, 1H), 3.96 (m, 1H), 3.96 (d, J=3.5 Hz, 1H), 4.12 (q, J=6.6 Hz, 2H), 5.28 (d, J=3.5 Hz, 1H), 6.59-6.65 (m, 1H), 6.67-6.75 (m, 1H), 6.86-6.94 (m, 2H), 7.26-7.41 (m, 5H).

¹³C NMR (100.62 MHz, CDCl₃) δ 14.76, 62.02, 74.28, 86.30, 112.37, 116.09, 120.72, 122.34, 126.33, 128.12, 128.75, 137.85.

### Examples 3 and 4

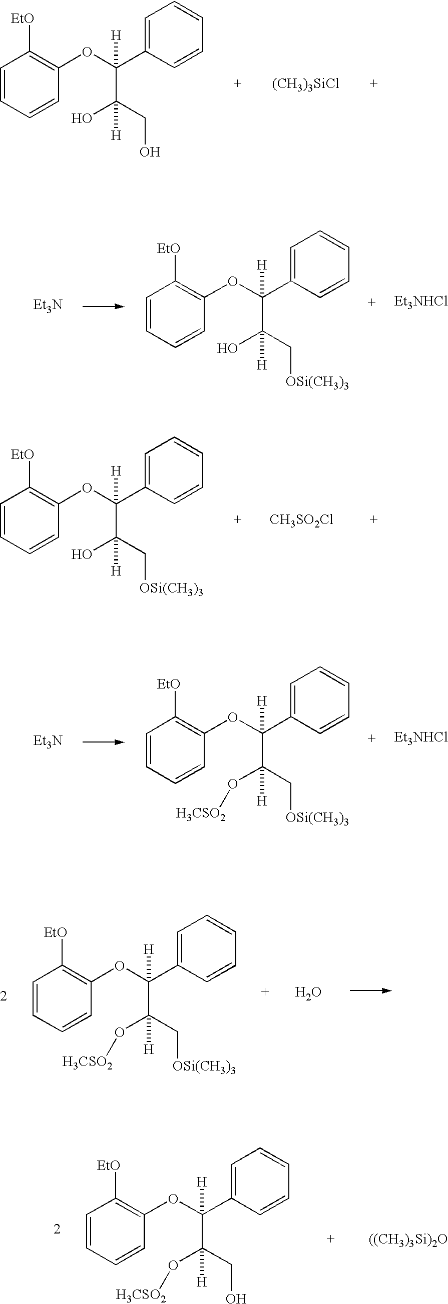

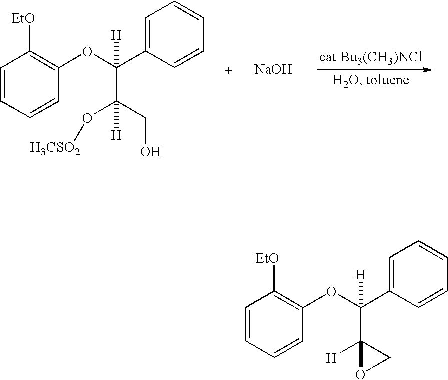

### Example 3

### (2R,3S)-3-(2-Ethoxyphenoxy)-2-mesyloxy-3-phenyl-1-hydroxypropane

(2R,3S)-3-(2-ethoxyphenoxy)-2-hydroxy-3-phenylpropanol (40.00 g) and triethylamine (23.4 mL) were dissolved in methylene chloride (400 mL) and the solution was cooled to about −20° C. A solution of trimethylsilyl chloride (18.7 mL) in CH₂Cl₂ (28 mL) was added keeping the internal temperature less than −15° C. After the addition was complete, the mixture was stirred for about 15 minutes at less than −15° C. Methanesulfonyl chloride (13.2 mL) was added to the solution, keeping the temperature between −20 and −15° C., followed by triethylamine (19.5 mL), again maintaining a pot temperature between −20 and −15° C. The mixture was stirred for 15 minutes after completion of triethylamine addition. Hydrochloric acid (1 M, 140.6 mL) was added to the reaction mixture. The mixture was warmed to 20-25° C. and stirred for 12 h. The phases were separated and the organic solution was washed with 5% (w/v) aqueous sodium bicarbonate solution (131 mL). The aqueous phase was separated to give a solution of the title compound.

### Example 4

### (2S,3S)1,2-Epoxy-3-(2-ethoxyphenoxy)-3-phenylpropane

Sodium hydroxide (25 g), tetra-n-butylammonium chloride (1.92 g), and water (100 mL) were stirred until the solids were dissolved. The solution of (2R,3S)-3-(2-ethoxyphenoxy)-2-mesyloxy-3-phenyl-1-hydroxypropane from Example 3 in methylene chloride was added and the mixture was stirred at a high rate until the reaction was complete. The phases were separated and the aqueous phase was extracted with methylene chloride (100 mL). The combined organic phases were washed with saturated aqueous NaCl solution (76 mL). The organic solution was concentrated under vacuum to 60 mL. Methanol (300 mL) was added and the solution was concentrated to 60 mL. Methanol (300 mL) was added and the mixture distilled to a volume of 60 mL to give a solution of the title compound.

### Example 5

### (2S,3S)-3-(2-Ethoxyphenoxy)-2-hydroxy-3-phenylpropylamine

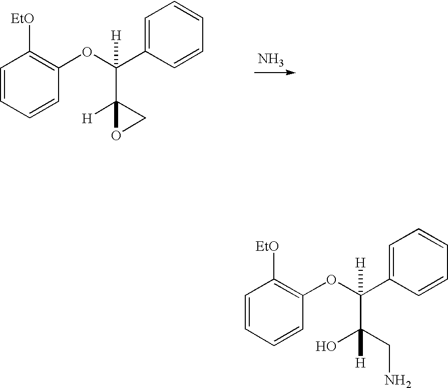

The solution of (2S,3S)-1,2-epoxy-3-(2-ethoxyphenoxy)-3-phenylpropane from Example 4, methanol (280 mL), and concentrated ammonium hydroxide (450 mL) were charged to a 1 L pressure flask equipped with a magnetic stirrer. The flask was sealed and heated to 40° C. for three hours. The mixture was cooled and methylene chloride (340 mL) was added. The phases were separated and the aqueous phase was extracted with methylene chloride (2×150 mL). The organic phases were combined and distilled to a volume of 450 mL. Methylene chloride (250 mL) was added back to the product solution. The organic phase was washed with water (375 mL), the aqueous phase was extracted with methylene chloride (150 mL) and the methylene chloride solutions were combined. Hydrochloric acid (0.5 M, 375 mL) was added and the mixture was stirred and then allowed to settle. The phases were separated and the organic phase was washed with water (375 mL). The aqueous phases were combined and washed with methylene chloride (70 mL). The organic phase was separated and discarded. Methylene chloride (220 mL) was added and then 50% NaOH solution was added until the pH was greater than 12. The phases were separated and the aqueous phase was extracted with methylene chloride (100 mL). The organic phases were combined and distilled to a solid. Ethyl acetate (2×250 mL) was added and distilled. Ethyl acetate (116 mL) and heptane (116 mL) were added. The mixture was heated until the solids dissolved. The solution was cooled slowly to 20-25° C. with rapid stirring. When the temperature of the slurry reached 20-25° C., heptane (116 mL) was added and the slurry was cooled to −15° C. and held at 15° C. for 1 hour. The solids were filtered and dried on a nitrogen press to yield 25 g of the title compound.

mp 98.5-99.9° C.

[α]²⁰_{D} (c=10)+37.04°

¹H NMR (400.13 MHz, CDCl₃) δ 1.51 (t, J=7.1 Hz, 3H), 2.5-2.7 (m, 4H), 3.94 (m, 1H), 4.11 (q, J=7.1 Hz, 2H), 4.75 (d, J=8.1 Hz, 1H), 6.62-6.86 (m, 4H), 7.23-7.40 (m, 5H).

¹³C NMR (100.62 MHz, CDCl₃) δ 14.83, 43.11, 64.21, 87.77, 112.84, 119.58, 120.82, 123.32, 127.25, 128.60, 148.06.

1. A method of preparing a compound of formula (IV): 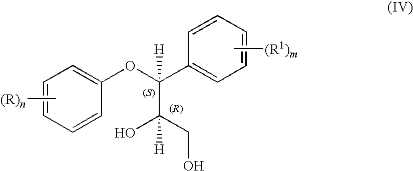
wherein
n and m are, independently, 0 or an integer from 1 to 5;
each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆alkyl wherein the aryl ring is optionally substituted by one or more substituents selected from C₁-C₆ alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆alkoxy wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical;
comprising:
(a) asymmetric epoxidation of a compound of formula (I): 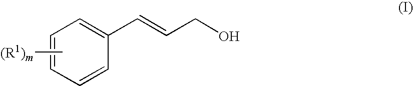
with an oxidising agent in the presence of an optically active compound, to give a compound of formula (II): 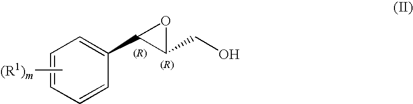
followed by
(b) reaction of the compound of formula (II) with a compound of formula (III): 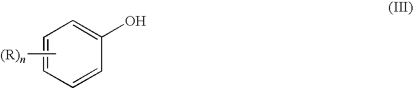
in the presence of a base;
wherein the method is carried out without isolation of the compound of formula (II).

2. A method of preparing a compound of formula (IX): 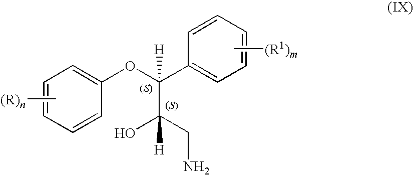
wherein
n and m are, independently, 0 or an integer from 1 to 5;
each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆ alkyl wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆alkoxy wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical;
comprising:
(a) preparing a compound of formula (IV) according to the method of claim 1;
(b) reaction of the compound of formula (IV) with a silylating agent to produce a compound of formula (V): 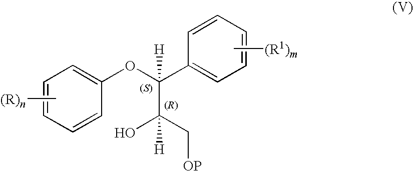
P is a silyl protecting group;
(c) reaction of the compound of formula (V) with a sulfonylating agent of formula R′SO₂X, wherein R′ is a sulfonic acid residue and X is a leaving group, to produce a compound of formula (VI): 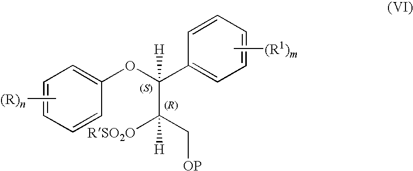
P is as defined above, and R′ is a sulfonic acid residue;
(d) removal of the silyl protecting group to produce a compound of formula (VII): 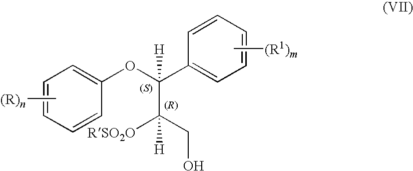
(e) displacement of the sulfonyloxy group to give a compound of formula (VIII): 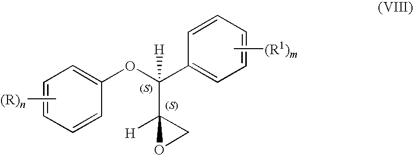
and
(f) reaction of the compound of formula (VIII) with ammonia or an ammonium compound to give the compound of formula (IX).

3. A method according to claim 2, wherein the method is carried out without isolation of the compounds of formulae (V), (VI), (VII) and (VIII).

4. A method according to claim 1, wherein R is ethoxy.

5. A method according to claim 1, wherein n is 1 and m is 0.

6. A method according to claim 1, wherein n is l, m is 0 and R is ethoxy at the 2-position of the phenyl ring.

7. A method according to claim 1, wherein the oxidising agent used in step (a) is a hydroperoxide.

8. A method according to claim 9, wherein the oxidising agent used in step (a) is t-butyl hydroperoxide.

9. A method according to claim 1, wherein the optically active compound used in step (a) is (−)-diisopropyl tartrate.

10. A method according to claim 1, wherein step (a) is carried out in the presence of titanium isopropoxide.

11. A method according to claim 1, wherein following completion of step (a), a quenching agent is added in order to quench any excess oxidising agent present.

12. A method according to claim 11, wherein the quenching agent is triethyl phosphite.

13. A method according to claim 1, wherein step (b) is carried out under phase transfer conditions.

14. A compound of formula (V): 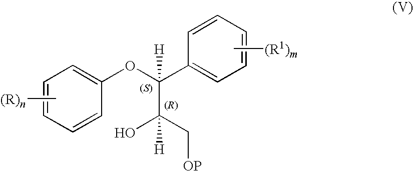
wherein
n and m are, independently, 0 or an integer from 1 to 5;
each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆ alkyl wherein the aryl ring is optionally substituted by one or more substituents selected from C₁-C₆ alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆ alkoxy wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical;
and P is a silyl protecting group.

15. A compound of formula (VI): 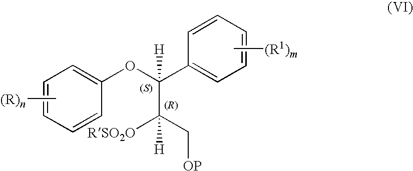
wherein
n and m are, independently, 0 or an integer from 1 to 5;
each of the groups R and R¹, which may be the same or different, is halogen; halo-C₁-C₆alkyl; hydroxy; C₁-C₆alkoxy; C₁-C₆alkyl optionally substituted by one or more substituents selected from halogen, hydroxy, C₁-C₆alkoxy, NR⁵R⁶ wherein R⁵ and R⁶ are, independently, hydrogen or C₁-C₆ alkyl, or —CONR⁵R⁶ wherein R⁵ and R⁶ are, independently, hydrogen or C₁-C₆ alkyl; aryl-C₁-C₆ alkyl wherein the aryl ring is optionally substituted by one or more substituents selected from C₁-C₆alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; aryl-C₁-C₆ alkoxy wherein the aryl ring is optionally substituted by one or more substituents selected from C₁-C₆ alkyl, halogen, halo-C₁-C₆alkyl, hydroxy, C₁-C₆alkoxy, and NR⁵R⁶ wherein R⁵ and R⁶ are, independently, hydrogen or C₁-C₆ alkyl; —NO₂; NR⁵R⁶ wherein R⁵ and R⁶ are, independently, hydrogen or C₁-C₆ alkyl, or two adjacent R groups or two adjacent R¹ groups, taken together, form a —O—CH₂—O— radical;
and P is a silyl protecting group and R′ is a sulfonic acid residue.
